(19) United States
(12) Patent Application Publication
(10) Pub. No.: US 2009/0246848 A1
(43) Pub. Date: Oct. 1, 2009

(54) PROCESS AND APPARATUS FOR DEWATERING CELLULOSIC FERMENTATION PRODUCTS

(76) Inventor: Gaetan Noel, St-Hubert (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/415,117

(22) Filed: Mar. 31, 2009

Related U.S. Application Data
(60) Provisional application No. 61/041,342, filed on Apr. 1, 2008.

(51) Int. Cl. C12P 7/10; C12M 1/12

(52) U.S. Cl. 435/165; 435/297.1

(57) ABSTRACT

A liquid mixture of water and a small percentage of an alcohol, for example a cellulosic fermentation broth, is converted into a mixture of vapours. The vapour mixture includes an increased percentage of alcohol vapour relative to the liquid mixture but is mostly water vapour. Water vapour is removed from the vapour mixture by permeation through a vapour separation membrane unit. Retained vapour has an increased alcohol content, optionally to the level of a fuel grade alcohol. Heat energy in permeate or product vapours or both may be recovered, for example by us as heating steam or by flow through a heat exchanger. The membrane unit may have two or more stages. Permeate from a stage may be condensed and used for example as fermentation make up water, compressed and fed to the permeate from an upstream stage or heating steam, or fed to another membrane stage for further dewatering.

This application claims the benefit under 35 USC 119(e) of U.S. patent application Ser. No. 61/041,342 filed on Apr. 1, 2008, which is incorporated herein in its entirety by this reference to it.

### FIELD

This specification relates to processing, producing or dewatering fermentation products, for example acetone, butanol, ethanol or mixtures, or membrane vapour separation.

### BACKGROUND

The following is not an admission that anything discussed herein is prior art or common knowledge of persons skilled in the art.

Plant matter including cellulosic or ligno-cellulosic materials may be treated by hydrolysis or saccharification and fermentation to produce a liquid broth, sometimes called beer, which is primarily water but includes one or more alcohols. A broth may be produced having up to about 7 or 8 wt % ethanol but the productivity of the fermentation yeasts decreases as the concentration of ethanol in the broth rises. For example, at an ethanol concentration of only 5 wt % in the broth, productivity of the yeasts will have declined by about 30% compared to their uninhibited productivity. Accordingly, cellulosic fermentation processes are operated to produce a broth having from 1-5 wt % ethanol, typically about 3 wt %.

In order to produce a higher alcohol content product, useful for example as fuel, the broth must be substantially dehydrated. For example, fuel grade ethanol in North American or other cold climate markets must have at least 99 wt % ethanol. Current fuel grade ethanol plants typically dewater fermentation broths by a combination of distillation to roughly 90 wt % ethanol followed by further dewatering with molecular sieves. When a broth is made by fermenting corn starch or sugar cane, the ethanol content of the broth may be in the range of 10-15 wt % ethanol. Dewatering that broth with distillation and molecular sieves uses about 5 MJ/L of fuel grade ethanol produced which is acceptable given that heat of combustion of ethanol of about 23 MJ/L. However, the energy required to dewater a cellulosic fermentation broth from about 3 wt % ethanol using distillation and molecular sieve is much higher, over 9 MJ/L of fuel grade ethanol produced. This represents a significant percentage of the heat of combustion of the ethanol produced and, when combined with other energy inputs needed in the complete production cycle, makes cellulosic processes with conventional dehydration less attractive.

### INTRODUCTION

The following introduction is not intended to limit or define any claim. One or more inventions may reside in any combination of one or more process steps or apparatus elements drawn from a set of all process steps and apparatus elements described below or in other parts of this document, for example the detailed description, claims or figures.

This specification describes an apparatus or process for removing water from a mixture comprising a low amount of an alcohol, for example 8 wt % or less or 5 wt % or less, such as a cellulosic fermentation broth. A vaporization process, for example evaporation, stripping or (partial) distillation, is used to produce a mixture of vapours from the broth having an alcohol content below about 40 or 45 wt %, for example 15-35 wt %. The vapour mixture is further processed in a gas separation membrane unit. A permeate is produced from the membrane unit that is substantially water vapour. This water vapour is compressed and used to transfer heat to one or more other parts of the process, for example the vaporization process, or for other uses, for example drying stillage or pre-heating vapours before membrane separation. The water vapour may also be condensed and used in the process, for example as make up water for fermentation. A retained (non-permeated) vapour from the membrane unit contains enriched alcohol, optionally dehydrated to fuel grade standards. Heat energy in the retained product vapour may also be used, for example to dry stillage or pre-heat the broth before vaporization.

The membrane unit may have multiple gas separation membrane stages, for example two, three or more. The stages may be arranged in series in relation to a feed/retentate/product flow. Permeate from an upstream stage may be compressed and used to heat another process step. Permeate from a downstream stage may be condensed for use as make up water, or compressed and added to an upstream permeate stream. A permeate stream may be fed through another membrane stage for further dewatering before being used to transfer heat. One or more of the possibilities described above may be combined.

### BRIEF DESCRIPTION OF THE FIGURES

FIG. 1 is a simplified schematic flow sheet of an ethanol processing plant.

FIG. 2 is a simplified schematic flow sheet of a scrubber of the plant of FIG. 1.

FIGS. 3 to 6 are simplified schematic flow sheets of alternate membrane units of the plant of FIG. 1.

FIG. 7 is a flow sheet of the distillation and dehydration section of an ethanol plant using molecular sieves provided as the background for a comparative example.

FIG. 8 is a flow sheet of the distillation and dehydration section of an ethanol plant using vapour separation membranes rather than the molecular sieves of FIG. 7.

### DETAILED DESCRIPTION

Various apparatuses or processes will be described below to provide an example of an embodiment of each claimed invention. No embodiment described below limits any claimed invention and any claimed invention may cover processes or apparatuses that are not described below. The claimed inventions are not limited to apparatuses or processes having all of the features of any one apparatus or process described below or to features common to multiple or all of the apparatuses described below. It is possible that an apparatus or process described below is not an embodiment of any claimed invention. The applicants, inventors and owners reserve all rights in any invention disclosed in an apparatus or process described below that is not claimed in this document and do not abandon, disclaim or dedicate to the public any such invention by its disclosure in this document.

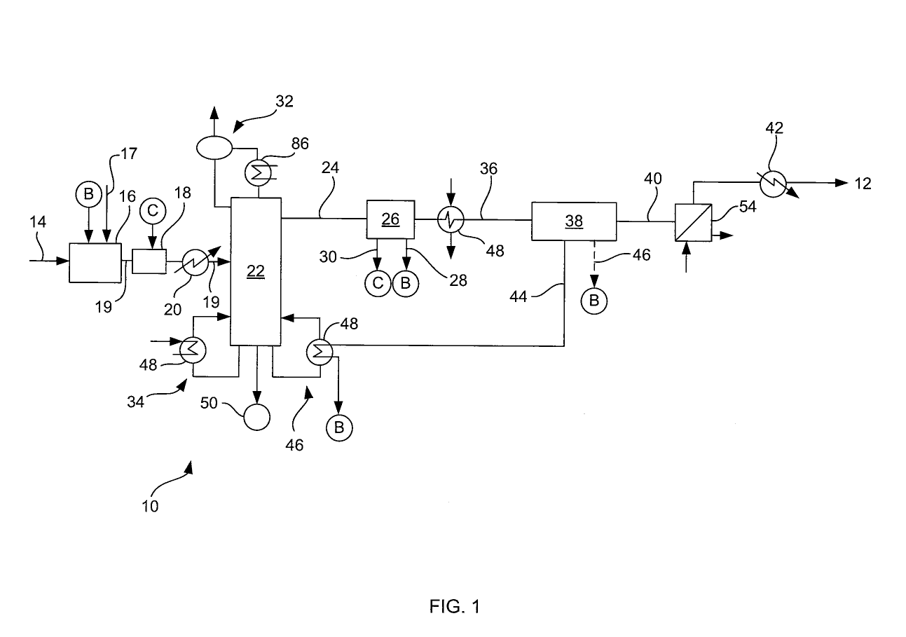

FIG. 1 shows a plant 10 used to produce a product 12. The product 12 is a substantially anhydrous alcohol or mixture of alcohols. For example, the product 12 may be fuel grade alcohol, for example 99 wt % or more ethanol. The raw feed 14 to the plant 10 is a plant material that may be fermented to produce an alcohol, for example carbohydrates or cellulose or lingo-cellulose, for example from corn kernels, wheat, sugarcane, switchgrass or agricultural, forest or paper-making waste products. The raw feed 14 passes to a fermenter 16 which is also fed with water 17 as well as yeast and other fermentation inputs. Where the feed 14 is a cellulosic or lingo-cellulosic material, the feed 14 may pass through a hydrolosis step upstream of the fermenter 16 or the fermenter 16 may be a combined saccharification and fermentation reactor. The fermenter 16 produces a beer 19 which may be temporarily stored in a beer tank 18. The beer 19 contains alcohol but is mostly water. Although higher alcohol contents can be achieved, the beer 19 typically contains about 5 wt % or less alcohol when cellulosic or lingo-cellulosic feedstock is used.

The beer 19 flows from the fermenter 16 to a vaporizing unit to produce a vapour mixture 24. In the plant 10 illustrated, the vaporizing unit is a distillation column 22. The beer 19 may pass through a beer pre-heater 20 on the way to the distillation column 22. The distillation column 22 may be or comprise a stripping column, optionally called a beer column. The distillation column 22 may have a reflux loop 32 and a reboiler loop 34.

While a beer column can be made to raise the alcohol content of the beer to a value in the range of 50-65 wt %, in the present invention the distillation column 22 or other vaporizing unit is preferably designed, selected or operated to produce a vapour mixture 24 with less than 45 wt % alcohol, for example 15-35 wt % alcohol. Since the required alcohol concentration is low, a simple or multi-stage evaporator may be used as the vaporization unit. The vaporaization unit also serves to separate solids in the beer from the vapour mixture 24.

The vapour mixture 24 may pass through a scrubber 26. Scrubber 26 will be described further below but removes particles and liquid droplets from the distilled ethanol 24. The particles are contained in a first liquid 28 which may be returned to the fermenter 16 as make up cook water and a second liquid 30 which may be returned to the beer tank 18.

Scrubbed vapour mixture 36 leaves the scrubber 26 and flows to the membrane unit 38. The membrane unit 38 will be described in further detail below. In general, the membrane unit 38 produces a product vapour 40 that is nearly water free. For example, in a plant 10 used to produce fuel grade ethanol for a cold climate market, the product vapour 40 my be 99 wt % or more ethanol. The membrane unit 38 also produces compressed vapour permeate 44 and, optionally, condensed permeate 46. Both permeates 44, 46 have only trace ethanol contents, for example 2% ethanol by volume or less. Condensed permeate 46, if any, may be returned to the fermenter 16 as make up cook water, or optionally sent to the distillation column 22. For reasons that will be discussed further below, compressed vapour permeate 44 carries heat energy and may be used to heat another part of the process. In FIG. 1, for example, the compressed vapour permeate 44 is used in a second reboiler loop 46 to heat the liquids in the bottom of distillation column 22. Optionally, condensed vapour permeate 44 may be used to replace or further supply heat to reboiler loop 34, beer preheater 20, a stillage dehydrator, a heater 48 or other apparatuses or processes. After transferring its heat energy, compressed vapour permeate 44 may become a liquid, primarily water, and be re-used, for example as make up cook water for fermenter 16.

Stillage 50 may be withdrawn from distillation column 22 or optionally from the beer feed to distillation column 22. Stillage 50 may be partially dewatered by mechanical means and then sent through a drying circuit for use, for example, as animal feed. Product vapour 40 may pass through one or more heat exchangers 54 to transfer energy to another process stream before passing through a condenser 42 to be converted into liquid product 12, for example essentially anhydrous ethanol.

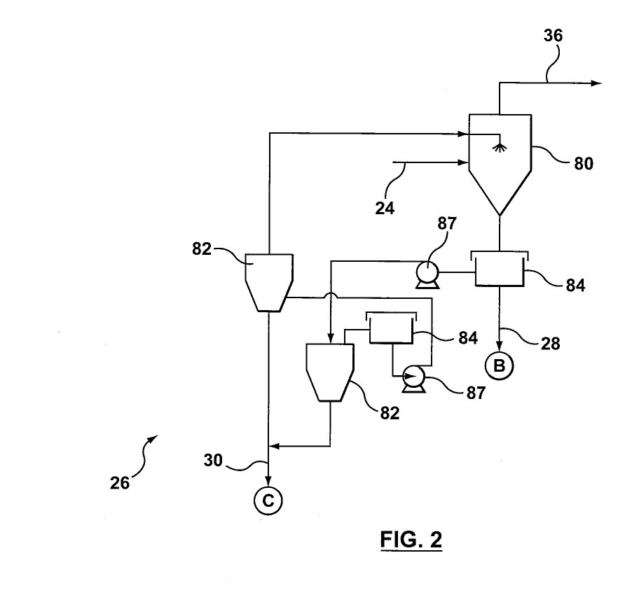

FIG. 2 shows scrubber 26 in greater detail. Scrubber 26 has a spray tank 80, tank 84, pumps 87, and forward cleaners 82 configured and connected as shown. Scrubber 26 removes particles and liquid droplets, if any, from the vapours by entraining the particles and droplets in water.

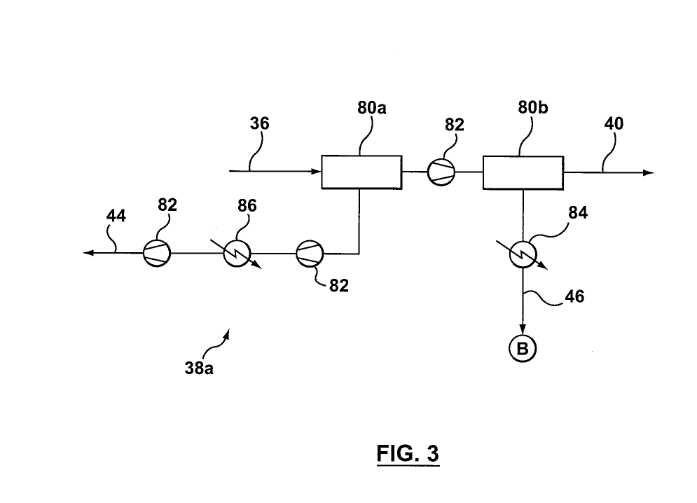

Various alternate membrane units 38 will be described below with reference to FIGS. 3 to 6. Each of FIGS. 3 to 6 show a different example of a membrane unit 38. Other examples of membrane units 38 may be created by combining all or parts of one or more of the examples of FIGS. 3 to 6. The membrane units 38 have multiple membrane stages 80. Each membrane stage 80 may be a membrane module, a stage in an internally staged module, or a set of modules or internal stages in parallel. Membrane modules may use polymeric membranes, for example of polyimide hollow fibers. A hollow fibre module may be fed to the insides of the hollow fibres. The membranes may be asymmetric integrally skinned polyimide membranes as described, for example, in International Patent Application No. PCT/CA2004/001047 filed on Jul. 16, 2004. Such membranes can have a vapour permeance for water of at least 1×10⁻⁷ mol/m²sPa at a temperature of about 30° C. to about 200° C., for example about 4×10⁻⁷ mol/m²sPa or more at about 80° C. The membrane may have a vapour permeance selectivity of at least 50, preferably at least 250 for water/ethanol at a temperature of about 140° C. Application No. PCT/CA2004/001047 and U.S. patent application Ser. Nos. 11/332,393 and 12/038,284 are incorporated herein in their entirety by this reference to them. The membrane unit 38 may comprise Siftek™ modules by Vaperma or as described in U.S. patent application Ser. No. 12/117,007 which is incorporated herein in its entirety by this reference to it.

The membrane unit 38 has a vapour compressor 82. The vapour compressor 82 compresses permeate vapours adiabatically which causes them to rise in temperature. The increased temperature allows the heat energy in the permeate vapours to flow to, and heat, lower temperature vapours, gases or liquids. The heat energy in the permeate vapours (sensible heat plus latent heat of condensation) can then be used for heating purposes in other parts of the process. The vapour compressor 82 may be, for example, a radial type fan or compressor that provides a compression ratio of less than 1:40, for example, between 1:2 and 1:10. Although the vapour compressor 82 requires energy to turn the compressor, at relatively low compression ratio the temperature rise of the vapours permits their use as a heat source, for example in a re-boiler.

FIG. 3 shows a two stage membrane unit 38a. Permeate from a first stage 80a is sent to a vapour compressor 82 and used as heating steam for distillation column 22 as described above. Retentate from the first stage 80a becomes feed for a second stage 80b. Permeate from second stage 80b passes through a condenser 84 before being reused as cook water for fermentation as described above. The use of a compressor 82 to increase the retentate pressure from first stage 80a is an option, or to compress permeate from the first stage 80a before it reaches a cooler 86.

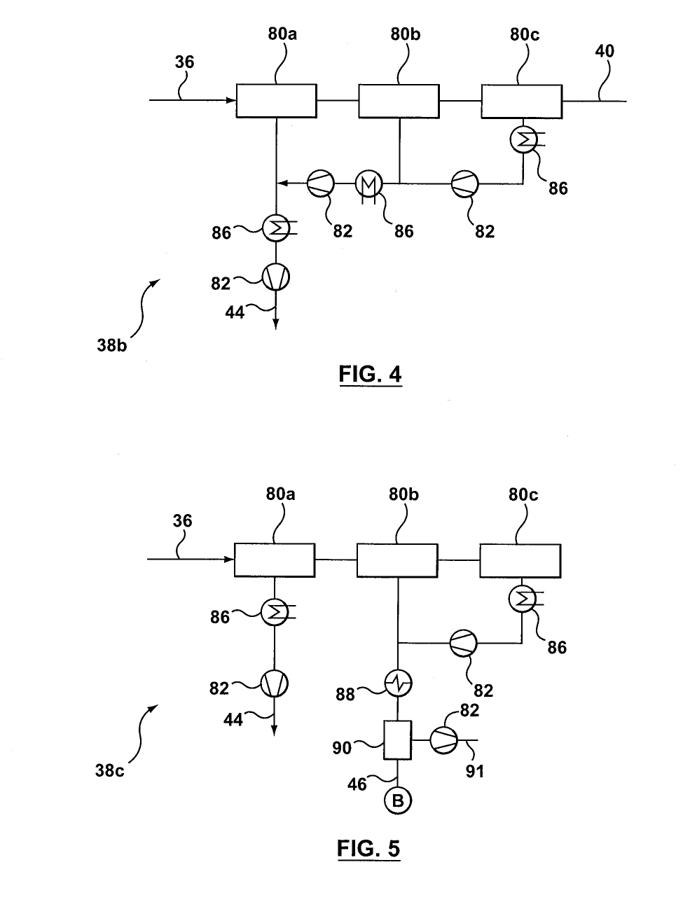

FIG. 4 shows a second membrane unit 38b having three stages 80a, 80b and 80c. Permeate from these stages 80a, 80b, 80c may have a temperature of about 100° C., but declining downstream, and pressures of about 30-60 kPa (absolute), 5-15 kPa (absolute) and 1.5 to 4 kPa (absolute) respectively. Optionally, the third stage 80c and its permeate flow may be omitted to create a two stage membrane unit. For each downstream unit 80b, 80c, the permeate is collected and passed through a cooler 86 and a vapour compressor 82 before joining the permeate from an adjacent upstream stage 80b upstream of its vapour compressor 82. Cooler 86 may assist in creating a permeate side vacuum to withdraw permeate and also allows the permeate vapour to be compressed to a higher pressure while contributing to reducing the outlet temperature of the compressed permeate. By recompressing permeate, and recycling it as heating steam, the second membrane unit 38b maximizes energy recovery. Compressed vapour permeate may have a temperature of 150° C. or more and a pressure of 200 kPa (absolute) or more.

FIG. 5 shows a third membrane unit 38c. The third membrane unit 38c combines aspects of the first membrane unit 38a and second membrane unit 38b. Two permeate streams 44, 46 are produced, but the condensed permeate 46 is produced from two downstream stages 80b, 80c connected with recycle and compression of the further downstream permeate to the adjacent upstream permeate as in the first membrane unit 38a. The combined permeate of downstream stages 80b, 80c passes through a condenser 88, and a holding tank 90 and is then recycled to the fermenter 16. The configuration of membrane unit 38c provides balanced cost and energy improvements. A compressor 82 connects the holding tank 90 to an outlet 91 to atmosphere.

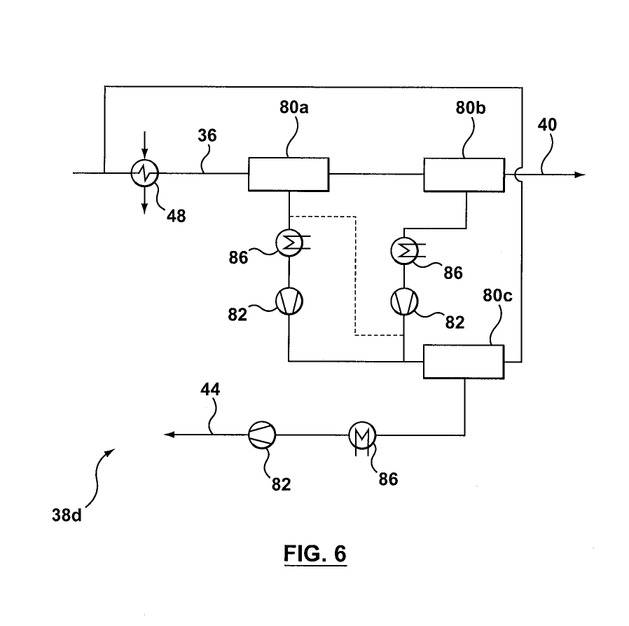

FIG. 6 shows a fourth membrane unit 38d. Permeate from first and second stages 80a, 80b is compressed and fed to third stage 80c individually as shown in the solid line or by joining the further downstream permeate to the adjacent upstream permeate before its compressor 82 as shown with the dashed line. Permeate from the third stage 80c is recycled upstream of the heater 48 upstream of the first stage 80a. Permeate vapour from the third stage is compressed and recycled as has been discussed above. In the third membrane unit 38d, the permeate is re-separated which increases ethanol recovery over the previous membrane units 38a, b, c. Compressed vapour permeate 44 may be 0.1% ethanol by volume or less, or essentially steam. Similarly, the permeate from any one or more stages described in FIGS. 3 to 5 may be further separated as shown in FIG. 6 to improve ethanol recovery.

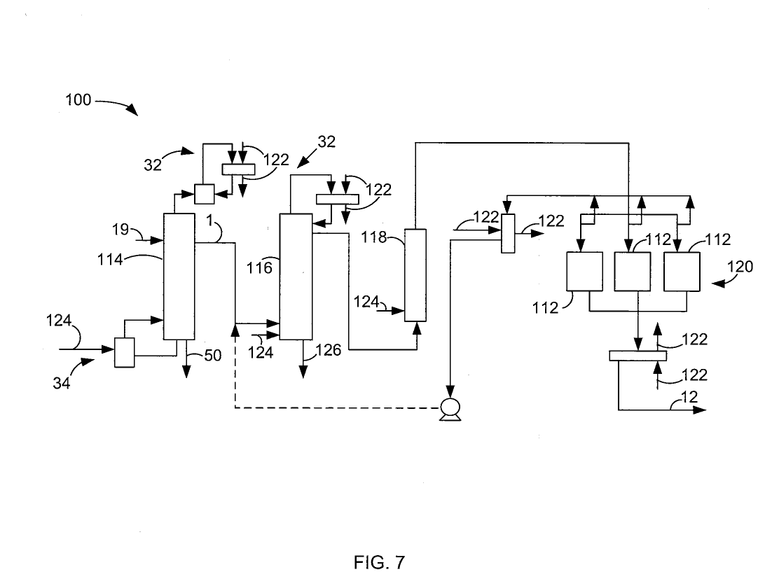

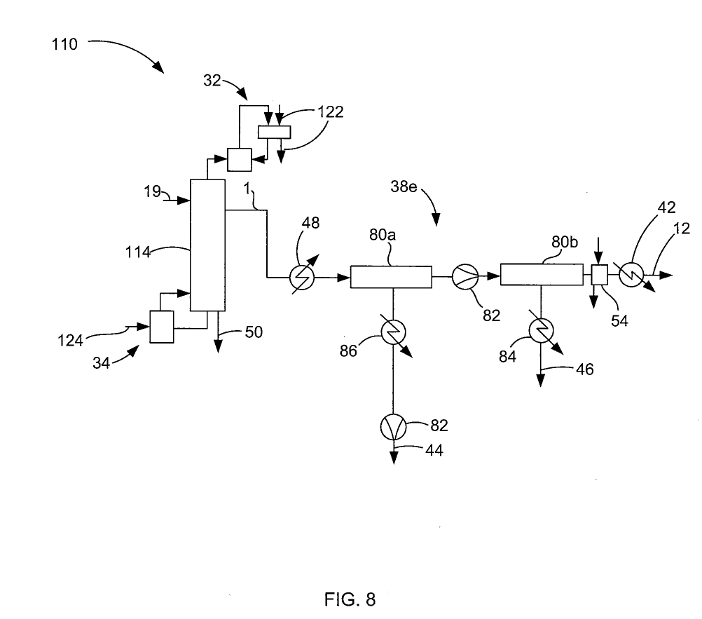

An example of a design application, shown in FIG. 8, using a membrane unit 38 will be compared below to a process, shown in FIG. 7, using stripping and rectification columns and molecular sieves. In the example, both plants are used to produce fuel grade ethanol. The examples show that the design of FIG. 8 reduces the process energy required compared to the design in FIG. 7. In FIG. 7 and 8, components similar to those described in previous figures are given the previously noted reference numerals.

FIG. 7 shows a process flow diagram of the distillation and dehydration sections 100 of a fuel ethanol plant with an upstream fermentor fed (not shown) and producing anhydrous ethanol at 99.2 wt %. The fermenter is fed with lingo-cellulosic materials and produces a broth with 3 wt % ethanol. The primary pieces of equipment in the distillation and dehydration sections 100 of the plant are a stripping column 114, a rectification column 116, an evaporator 118, and a pressure swing molecular sieve semi-continuous dehydration system 120 comprising three molecular sieve units 112. Stripping column 114 and rectification column 116 are parts of a two-column distillation unit. Cooling water 122 and steam 124 for heating are employed at various points in the distillation and dehydration sections 100. Product water 124 is also produced from the rectification column 116.

Broth from the fermenter into the stripping column 114 from which a stream at 50-65 EtOH w/w is extracted and directed into the rectification column 116. A condensed stream from the rectification column 116 is evaporated and pre-heated in the evaporator 118 prior to being fed into the molecular sieve system 120, from which dehydrated ethanol vapour is recovered and condensed afterwards as a 99.2% EtOH w/w product. The systems uses 9.21 MJ/I of thermal energy and 0.05 MJ/I of electrical energy for a total of 9.26 MJ/I.

An alternate distillation and dehydration section 110 using a membrane unit 38e to replace the rectification column 116, the evaporator 118 and the molecular sieve dehydration system 120 of FIG. 7 is shown in FIG. 8. The alternate distillation and dehydration section 110 of FIG. 8 reduces the energy demand of the distillation and dehydration operations compared to the distillation and dehydration section 100 of FIG. 7.

The membrane unit 38e replaces the rectification column 116, the evaporator 118 and the molecular sieve dehydration system 120 of FIG. 7, such that the distillation and dehydration section 110 of the ethanol plant now comprises two main processes and equipment units, the stripping column 114 and the membrane unit 38e. Further, the stripping column 114 is modified is to produce a vapour mixture of 25 wt % ethanol.

The membrane unit 38e comprises two membrane stages 80a, 80b in series, with a compressor 82 between, which raises the pressure of the retentate issued from the first stage 80a from about 110 kPa to about 225 kPa. Superheat may be recovered from the rententate upstream of the second membrane stage 80b by adding an optional heat exchanger (not shown) in the retentate line between the stages 80a, 80b of the membrane unit 38e. Permeate from both stages 80a, 80b is essentially ethanol free and is condensed. Vapour permeate 44 from the first stage 80a is directed to a fermentation section of the plant after recovering heat from it as described previously. Condensed permeate 46 from the second stage 80b is directed back for re-distillation to the stripping column 114.

The system of FIG. 8 uses 8.05 MJ/l of thermal energy and 1.18 MJ/l of electrical energy but 5.65 MJ/l of thermal energy is recovered from the product and permeate vapours. For example, the reboiler loop 34 may be heated at least in part by exhausted steam in the form of vapour permeate 44 as described previously. Total energy use is 3.59 MJ/l, which is significantly below the energy use of the FIG. 7 system and the heat of combustion of ethanol.

While the examples above relate to producing fuel grade ethanol made from cellulosic fermentation for cold climate requirements, similar energy considerations also apply to ethanol dried to slightly higher water contents for warmer climate markets, other fermentation products usable as fuels such as acetone, butanol or acetone, butanol, ethanol mixtures (ABE), and sugar cane, corn, wheat or other starch based fermentation processes where it is desirable to have a product concentration in the broth of less than about 8% or less than about 5%, for example to reduce product inhibition, to account for low sugar concentration in the feed, to reduce enzyme inhibition or to limit the concentration of suspended solids in the fermenter.

While various examples of devices or processes have been described above, various other specific devices or processes may also be within the scope of the invention defined by the following claims.

I claim:

1. A process for producing a substantially anhydrous fermentation product comprising the steps of,
a) treating one or more plant derived materials to produce a broth having 5 wt % or less of the fermentation product;
b) extracting a vapour mixture from the broth, the vapour mixture having 45 wt % or less of the fermentation product;
c) removing water vapour from the vapour mixture through a vapour separation membrane to produce a product vapour; and,
d) extracting heat energy from one or more of the removed water or product vapour.

2. The process of claim 1 wherein the vapour mixture extracted from the broth has 35 wt % of the fermentation product or less.

3. The process of claim 1 wherein the plant material comprises cellulosic or ligno-cellulosic material.

4. The process of claim 1 further comprising a step of compressing at least some of the removed water vapour and using heat carried by the removed water vapour to assist in distilling the mixture.

5. The process of claim 1 further comprising compressing removed water vapour and recycling it to a fermenter.

6. The process of claim 1 further comprising collecting product vapour from the membrane and passing it through a heat exchanger.

7. The process of claim 1 wherein the step of extracting a vapour mixture from the broth is performed in a single distillation column.

8. A process for producing a substantially anhydrous fermentation product comprising the steps of,
a) treating one or more plant derived materials, wherein the one or more plant derived materials comprise cellulosic or ligno-cellulosic material, to produce a broth;
b) extracting a vapour mixture from the broth, the vapour mixture having 45 wt % or less of the fermentation product;
c) removing water vapour from the vapour mixture through a vapour separation membrane to produce a product vapour; and,
d) extracting heat energy from one or more of the removed water or product vapour.

9. The process of claim 1 wherein the vapour mixture extracted from the broth has 35 wt % of the fermentation product or less.

10. The process of claim 1 further comprising a step of compressing at least some of the removed water vapour and using heat carried by the removed water vapour to assist in distilling the mixture.

11. The process of claim 1 further comprising compressing removed water vapour and recycling it to a fermenter.

12. The process of claim 1 further comprising collecting product vapour from the membrane and passing it through a heat exchanger.

13. The process of claim 1 wherein the step of extracting a vapour mixture from the broth is performed in a single distillation column.

14. An apparatus for producing a substantially anhydrous fermentation product comprising a cellulosic or ligno-cellulosic processing system, a broth vaporization unit and a membrane vapour separation unit, wherein these components are connected sequentially in a once-through feed to product flow path.
